(19) United States
(12) Patent Application Publication
(10) Pub. No.: US 2026/0033842 A1
(43) Pub. Date: Feb. 5, 2026

(54) ORTHOPEDIC RECIRCULATING SAW AND ORTHOPEDIC PLANER

(71) Applicant: Christopher PEDICINI, Brentwood, TN (US)

(72) Inventor: Christopher PEDICINI, Brentwood, TN (US)

(21) Appl. No.: 19/290,982

(22) Filed: Aug. 5, 2025

Related U.S. Application Data
(60) Provisional application No. 63/679,286, filed on Aug. 5, 2024.

(51) Int. Cl. A61B 17/14; A61B 17/00

(52) U.S. Cl. CPC A61B 17/142; A61B 17/149; A61B 2017/00367

(57) ABSTRACT

An orthopedic saw configured for orthopedic surgical procedures may include an element including a cutting surface, a drive mechanism, and a handle. The drive mechanism may be configured to move the cutting surface in a continuous recirculating motion. The handle may be configured to guide the cutting surface along a cutting path.

### CROSS-REFERENCE TO RELATED APPLICATION

This application claims the benefit of U.S. Provisional Application No. 63/679,286, filed Aug. 5, 2024, which is incorporated herein by reference in its entirety.

### BACKGROUND

Orthopedic surgical procedures, such as total knee arthroplasty (TKA), require precise bone cutting to achieve successful outcomes. During the surgical procedures, surgeons utilize orthopedic saws to resect problematic bone tissue from affected joints to prepare the affected joints to receive an implant.

### SUMMARY

In some aspects, the techniques described herein relate to an orthopedic saw configured for orthopedic surgical procedures, including: an element including a cutting surface; a drive mechanism configured to move the cutting surface in a continuous recirculating motion; and a handle configured to guide the cutting surface along a cutting path.

In some aspects, the techniques described herein relate to an orthopedic saw, including: an element including a curved surface and cutting teeth disposed along the curved surface; a drive mechanism configured to move the cutting teeth in a controlled motion; and a handle configured to guide the cutting teeth along a cutting path.

In some aspects, the techniques described herein relate to an orthopedic saw, including: an element including a cutting surface; a drive mechanism configured to move the cutting surface along a curved path; and a handle configured to guide the cutting surface along the curved path.

### BRIEF DESCRIPTION OF THE DRAWINGS

FIG. 1 is a diagram of an example orthopedic saw configured for orthopedic surgical procedures.

FIG. 2 is a diagram of an example orthopedic saw configured for orthopedic surgical procedures.

FIG. 3 is a diagram of an example orthopedic saw configured for orthopedic surgical procedures.

FIG. 4 is a diagram of an example orthopedic saw configured for orthopedic surgical procedures.

FIG. 5 is a diagram of an example orthopedic saw configured for orthopedic surgical procedures.

FIG. 6 is a diagram of an example orthopedic saw configured for orthopedic surgical procedures.

FIG. 7 is a diagram of an example orthopedic planer configured for orthopedic surgical procedures.

### DETAILED DESCRIPTION

The following detailed description of example implementations refers to the accompanying drawings. The same reference numbers in different drawings may identify the same or similar elements.

Sagittal saws are used in orthopedic surgical procedures. However, typical sagittal saws are associated with several challenges when used in the orthopedic surgical procedures. When using typical sagittal saws, surgeons must rely on guides (e.g., guide blocks and/or jigs) to facilitate cutting, which adds complexity to the orthopedic surgical procedures. Despite using the guides, an unintended phenomenon known as skiving (e.g., which is associated with blade deflection), can occur, which results in inaccurate and non-planar cuts. This problem can arise when a surgeon applies excessive force on the sagittal saw, which causes the blade to bend. Such excessive force might be necessary when the blade is not cutting efficiently or when the cutting process is too slow. Another significant issue occurs when the saw encounters relatively hard bone (e.g., sclerotic bone) during cutting. The relatively hard bone imparts reactionary forces on the sagittal saw, which causes the sagittal saw to deviate from an intended cutting path. Furthermore, the guides used with the typical sagittal saws obstruct a view of the surgeon during cutting.

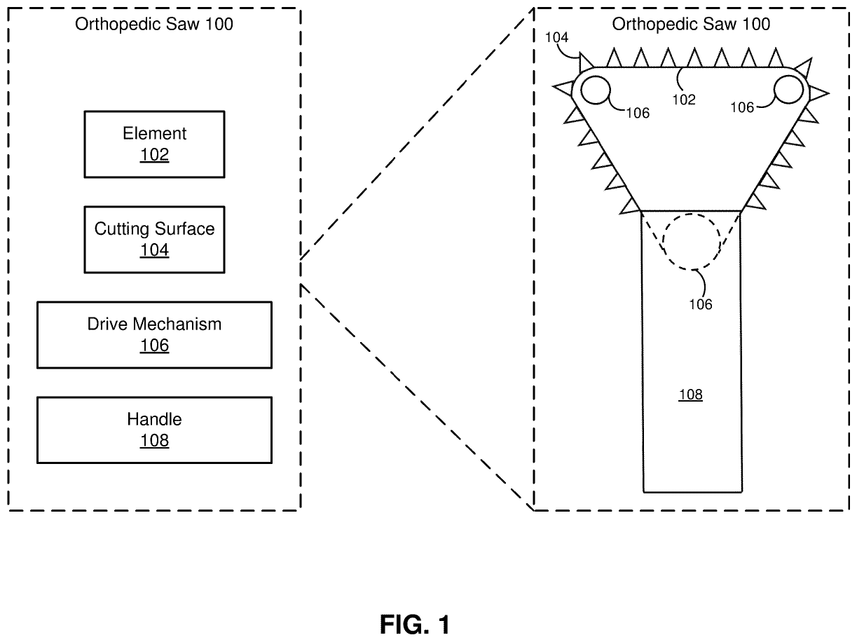

FIG. 1 is a diagram of an example, orthopedic saw 100 configured for orthopedic surgical procedures, as described herein. As shown in FIG. 1, the orthopedic saw 100 may include an element 102 including a cutting surface 104, a drive mechanism 106, and a handle 108.

In some implementations, the element 102 may be a flexible member. For example, the element may be implemented as a wire (e.g., an abrasive wire), a chain (e.g., a gigli chain), and/or a rope (e.g., an abrasive rope), among other examples. Accordingly, for example, the cutting surface 104 may be a part of the wire, the chain, and/or the rope.

In some implementations, the element may be disposable and configured for single-use per orthopedic surgical procedure. In some implementations, the drive mechanism 106 may be configured to move the cutting surface 104 in a continuous recirculating motion.

The orthopedic saw of claim 1, wherein the drive mechanism includes a variable speed control configured to vary a drive speed of the cutting surface 104. In some implementations, the continuous recirculating motion may include movement of the cutting surface 104 along a closed-loop path.

In some implementations, the handle 108 may be configured to guide the cutting surface 104 along a cutting path. As further shown in FIG. 1, the orthopedic saw 100 may include an optical device 110 configured to provide a visual cue, at a location forward of the cutting surface 104, indicative of an intended cutting path of the cutting surface 104.

As further shown in FIG. 1, the orthopedic saw 100 may include one or more sensors 112 configured to provide feedback when cutting deviates from a predetermined cutting plane.

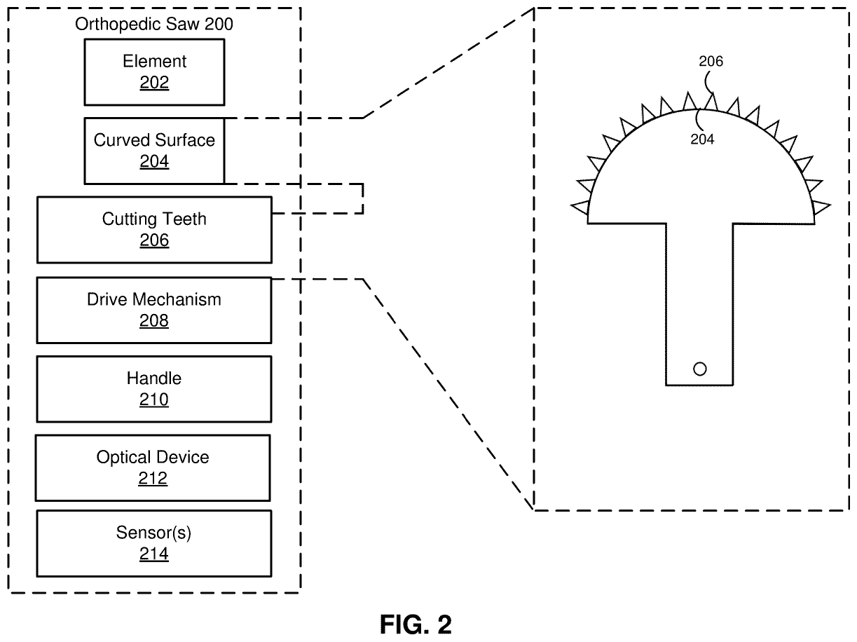

FIG. 2 is a diagram of an example orthopedic saw 200 configured for orthopedic surgical procedures, as described herein. As shown in FIG. 2, the orthopedic saw 200 may include an element 202 including a curved surface 204 and cutting teeth 206 disposed along the curved surface 204, a drive mechanism 208, and a handle 210.

In some implementations, the drive mechanism 208 may be configured to move the cutting teeth 206 in a controlled motion. In some implementations, the handle 210 may be configured to guide the cutting teeth 206 along a cutting path. In some implementations, the controlled motion may be an oscillatory motion and/or a reciprocating motion. In some implementations, the controlled motion may follow a circular path and/or an elliptical path.

In some implementations, a frequency of the controlled motion may be associated with a range. For example, the frequency of the controlled motion may be between approximately 15,000 hertz and approximately 20,000 hertz, among other examples (e.g., to allow the cutting teeth 206 to cut bone while preventing the cutting teeth 206 from cutting tissue in proximity to the bone, among other examples).

In some implementations, a displacement of the cutting teeth 206 may be associated with a distance (e.g., a total distance or a maximum distance). For example, the total displacement of the cutting teeth 206 may be approximately 0.5 inches or less, among other examples.

In some implementations, the drive mechanism 208 may include a variable speed control configured to vary a drive speed of the cutting teeth 206. As further shown in FIG. 2, the orthopedic saw 200 may include an optical device 212 configured to provide a visual cue, at a location forward of the cutting teeth 206, indicative of an intended cutting path of the cutting 206. As further shown in FIG. 2, the orthopedic saw 200 may include one or more sensors 214 configured to provide feedback when cutting deviates from a predetermined cutting plane.

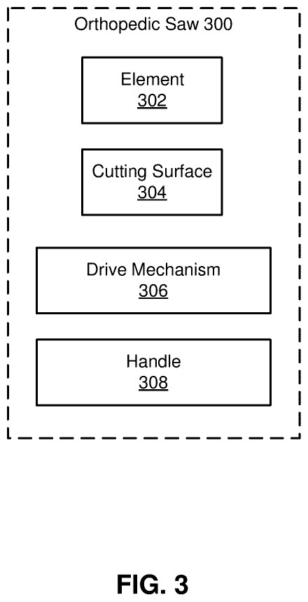

FIG. 3 is a diagram of an example orthopedic saw 300 configured for orthopedic surgical procedures, as described herein. As shown in FIG. 3, the orthopedic saw 300 may include an element 302 including a cutting surface 304, a drive mechanism 306, and a handle 308.

In some implementations, the drive mechanism may be configured to move the cutting surface along a curved path. In some implementations, the handle 308 may be configured to guide the cutting surface along the curved path. In some implementations the curved path may be associated with an excursion angle that is greater than 8 degrees.

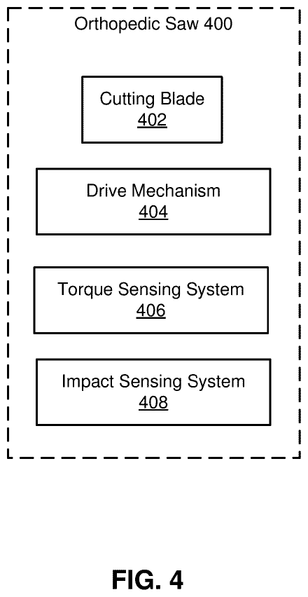

FIG. 4 is a diagram of an example orthopedic saw 400 configured for orthopedic surgical procedures, as described herein. As shown in FIG. 4, the orthopedic saw 400 may include a cutting blade 402, a drive mechanism 404 configured to drive the cutting blade 402, a torque sensing system 406 configured to detect when a torque associated with driving the cutting blade 402 exceeds a threshold (e.g., a predetermined threshold torque), and an impact activation system 408 configured to activate (e.g., automatically activate) an impact mode when the torque sensing system 406 detects the torque exceeding the threshold. In some implementations, the impact mode may be associated with applying impact forces (e.g., intermittent impact forces) to the cutting blade 402, such as in a plane parallel to the cutting blade 402, among other examples.

In some implementations, the threshold torque may be approximately 4 inch-pounds. In some implementations, the impact forces may be applied when the cutting blade 402 contacts a cutting area, such as a bone of a patient, among other examples. In some implementations, the impact mode may be configured to overcome resistance, such as resistance from dense bone material. In some implementations, the torque sensing system 406 may be configured to monitors (e.g., continuously monitor) torque during cutting operations (e.g., performed during the orthopedic surgical procedures).

In some implementations, the impact activation system 408 may be configured to deactivate (e.g., automatically deactivate) when the detected torque falls below the threshold. In some implementations, the impact forces may be applied in a direction substantially parallel to a cutting surface of the cutting blade 402. In some implementations, the orthopedic saw 400 may include a control system configured to adjust the threshold based on one or more conditions (e.g., one or more cutting conditions, among other examples).

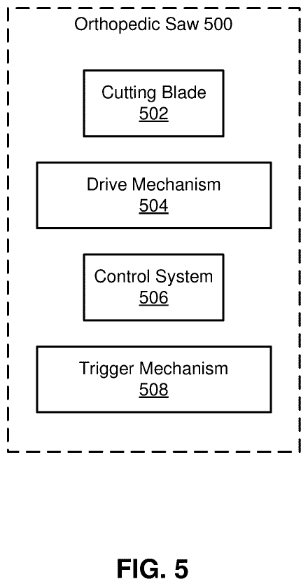

FIG. 5 is a diagram of an example orthopedic saw 500 configured for orthopedic surgical procedures. As shown in FIG. 5, the orthopedic saw 500 may include a cutting blade 502, a drive mechanism 504 configured to drive the cutting blade 502, and a control system 506 configured to implement a soft start mode.

In some implementations, the control system 506 may be configured to increase a speed of the cutting blade 502 from a first operating speed (e.g., an initial operating speed) to a second speed (e.g., a maximum operating speed), such as over a time interval (e.g., a predetermined ramp-up time period). For example, the predetermined ramp-up time period may be greater than 100 milliseconds and less than 1 second, among other examples. As another example, the predetermined ramp-up time period may be between approximately 200 milliseconds and approximately 800 milliseconds, among other examples. In some implementations, the first operating speed may be a zero, or a substantially zero, operating speed.

In some implementations, an increase in speed from the first operating speed to the second operating speed may follow a linear ramp profile during the predetermined ramp-up time period. In some implementations, an increase in speed from the first operating speed to the second operating speed may follow a curved ramp profile during the predetermined ramp-up time period.

In some implementations, the control system 506 may be configured to adjust the predetermined ramp-up time period based on one or more parameters, such as one or more user settings, among other examples. In some implementations, the soft start mode may be activated (e.g., automatically activated) when the cutting blade 502 is initially energized.

In some implementations, the soft start mode may allow a surgeon to position the cutting blade 502 against a cutting area (e.g., a bone tissue, among other examples) before the cutting blade 502 reaches the second operating speed.

In some implementations, the control system 506 may be configured to maintain alignment of the cutting blade 502 with an intended cutting plane (e.g., by preventing movement of the cutting blade 502 based on one or more conditions). In some implementations, the orthopedic saw 500 may include a trigger mechanism 508 and the soft start mode may be configured to be activated in response to the trigger mechanism being activated. In some implementations, the soft start mode may allow enhanced cutting accuracy by reducing initial blade movement forces.

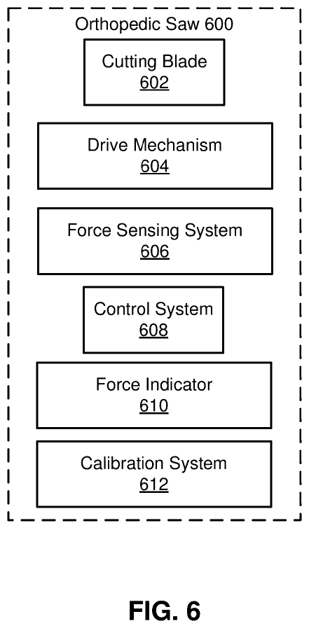

FIG. 6 is a diagram of an example orthopedic saw 600 configured for orthopedic surgical procedures. As shown in FIG. 6, the orthopedic saw 600 may include a cutting blade 602, a drive mechanism 604 configured to drive the cutting blade 602, a force sensing system 606 configured to detect a force (e.g., applied by an operator of the orthopedic saw 600) to engage the cutting blade 602 against a cutting area (e.g., a bone of a patient, among other examples), and a control system 608 configured to activate the cutting blade 602 when the detected force exceeds a first threshold (e.g., a minimum force threshold) and limit operation of the cutting blade 602 when the detected force exceeds a second threshold (e.g., a maximum force threshold) to prevent deflection of the cutting blade 602.

In some implementations, the first threshold may be approximately 1 pound. In some implementations, the second threshold force may be approximately 10 pounds. In some implementations, the first threshold force and the second threshold force may be based on one or more conditions, such as one or more conditions associated with the cutting blade 602, among other examples. In some implementations, the orthopedic saw 600 may include a force indicator 610 configured to provide feedback to the surgeon regarding the detected force. For example, the force indicator may include an audio indicator, a visual indicator, and/or a tactile feedback indicator.

In some implementations, the control system 608 may be configured to reduce an operating speed of the cutting blade 602 when the detected force approaches the second threshold. In some implementations, the control system 608 may be configured to deactivate the cutting blade 602 when the detected force exceeds the second threshold.

In some implementations, the force sensing system 606 may be configured to monitor (e.g., continuously monitor) the applied force during cutting operations. In some implementations, the control system 608 may be configured to facilitate accurate cuts by controlling an operating speed of the cutting blade 602 in association with force limitations.

As further shown in FIG. 6, the orthopedic saw 600 may include a calibration system 612 configured to adjust the first threshold and/or the second threshold based on one or more conditions, such as one or more conditions associated with the cutting blade 602, among other examples.

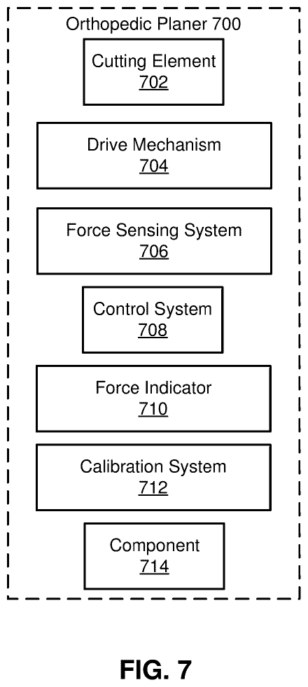

FIG. 7 is a diagram of an example orthopedic planer 700 configured for orthopedic surgical procedures. As shown in FIG. 7, the orthopedic planer 700 may include a cutting element 702 configured to plane a surface (e.g., a surface of a bone, among other examples), a drive mechanism 704 configured to move the cutting element 702, and a handle 706 configured to guide the cutting element 702 along the surface (e.g., along the surface of the bone, among other examples).

In some implementations, the cutting element 702 may implemented as a rotatable cutting wheel including a blade (e.g., multiple blades) disposed on a circumferential surface of the cutting wheel. In some implementations, the drive mechanism 704 may be configured to rotate the cutting wheel at variable orientations, such as to facilitate surgical access.

In some implementations, a reference surface of the orthopedic planer 700 may be configured to lie flat against a guide block (e.g., shown as a guide block 708) affixed to the surface (e.g., the surface of the bone, among other examples). In this way, the reference surface may be configured to maintain a predetermined relationship between the cutting element 702 and the guide block 708, such as during planing operations associated with the orthopedic surgical procedures.

In some implementations, the orthopedic planer 700 may include a safety cover 710 configured to protect surrounding areas (e.g., surrounding tissue, among other examples) from the cutting element 702 while allowing the cutting element 702 to contact the surface (e.g., the surface of the bone, among other examples). In some implementations, the safety cover 710 may be configured to expose an intended cutting area associated with the surface (e.g., an intended cutting area associated with the surface of the bone, among other examples).

As further shown in FIG. 7, the orthopedic planer 700 may include a debris collection system 712 configured to capture (e.g., automatically capture) debris (e.g., bone shavings) generated during planing operations associated with the orthopedic surgical procedures.

In some implementations, the debris collection system 712 may include a component 714 (e.g., a debris bag) configured to sequester the debris (e.g., the bone shavings, among other examples). In some implementations, the cutting element 702 may be configured to smooth the surface (e.g., smooth the surface of the bone, among other examples) such that the surface is suitable for press-fit implant placement, among other examples. In some implementations, the orthopedic planer 700 may be configured for use after initial bone resection to refine bone surface quality, among other examples.

When an element is referred to herein as being “operatively coupled” to another element, it should be understood that the elements may interact and/or cooperate with one another in any suitable manner, such as to facilitate the performance of a function, an action, and/or an operation (e.g., either by one or both of the elements and/or by causing another element to perform the function, the action, and/or the operation, among other examples). For example, one or more elements may be connected and/or coupled to one another, with or without intervening elements, to enable the performance of the function, the action, and/or the operation.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

In the preceding specification, various example embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. An orthopedic saw configured for orthopedic surgical procedures, comprising:
an element including a cutting surface;
a drive mechanism configured to move the cutting surface in a continuous recirculating motion; and
a handle configured to guide the cutting surface along a cutting path.

2. The orthopedic saw of claim 1, wherein the continuous recirculating motion includes movement of the cutting surface along a closed-loop path.

3. The orthopedic saw of claim 1, wherein the element is a wire.

4. The orthopedic saw of claim 1, wherein the element is a chain.

5. The orthopedic saw of claim 1, wherein the element is a rope.

6. The orthopedic saw of claim 1, wherein the drive mechanism includes a variable speed control configured to vary a drive speed of the element.

7. The orthopedic saw of claim 1, further comprising:
an optical device configured to provide a visual cue, at a location forward of the element, indicative of an intended cutting path of the cutting surface.

8. The orthopedic saw of claim 1, further comprising:
one or more sensors configured to provide feedback when cutting deviates from a predetermined cutting plane.

9. The orthopedic saw of claim 1, wherein the element is disposable and configured for single-use per surgical procedure.

10. An orthopedic saw, comprising:
an element including a curved surface and cutting teeth disposed along the curved surface;
a drive mechanism configured to move the cutting teeth in a controlled motion; and
a handle configured to guide the cutting teeth along a cutting path.

11. The orthopedic saw of claim 10, wherein the controlled motion is an oscillatory motion.

12. The orthopedic saw of claim 10, wherein the controlled motion is a reciprocating motion.

13. The orthopedic saw of claim 10, wherein the controlled motion follows a circular path.

14. The orthopedic saw of claim 10, wherein the controlled motion follows an elliptical path.

15. The orthopedic saw of claim 10, wherein a frequency of the controlled motion is between approximately 15,000 hertz and approximately 20,000 hertz.

16. The orthopedic saw of claim 10, wherein a total displacement of the cutting teeth is approximately 0.5 inches or less.

17. The orthopedic saw of claim 10, wherein the drive mechanism includes a variable speed control configured to vary a drive speed of the cutting teeth.

18. The orthopedic saw of claim 10, further comprising:
an optical device configured to provide a visual cue, at a location forward of the element, indicative of an intended cutting path of the cutting teeth.

19. An orthopedic saw, comprising:
an element including a cutting surface;
a drive mechanism configured to move the cutting surface along a curved path; and
a handle configured to guide the cutting surface along the curved path.

20. The orthopedic saw of claim 19, wherein the curved path is associated with an excursion angle that is greater than 8 degrees.
